(19) United States
(12) Patent Application Publication
(10) Pub. No.: US 2016/0287190 A1
(43) Pub. Date: Oct. 6, 2016

(54) METHOD FOR GENERATING USER INTERFACE AND APPARATUS FOR GENERATING USER INTERFACE USING SAME

(71) Applicant: LG CNS CO., LTD., Seoul (KR)

(72) Inventor: Si Young PARK, Seoul (KR)

(73) Assignee: LG CNS CO., LTD.

(21) Appl. No.: 14/983,825

(22) Filed: Dec. 30, 2015

(30) Foreign Application Priority Data
Apr. 3, 2015 (KR) 10-2015-0047319

(51) Int. Cl. A61B 5/00; G06F 17/21; A61B 5/0205; G06F 3/0481

(52) U.S. Cl. CPC A61B 5/7425; G06F 3/0481; G06F 17/212; A61B 5/746; A61B 5/02055; A61B 5/7475

(57) ABSTRACT

Provide is a method and apparatus for generating a user interface for displaying patient health-related data. The method may include establishing a connection between a patient-side terminal and at least one measurement device, receiving, at the patient-side terminal, measured patient data from the at least one measurement device for at least one type of measurement related to patient health, setting, at the patient-side terminal, a level for the measured patient data based on data for a prescribed set of rules, displaying the measured patient data on a display screen, and changing a layout of the displayed patient data on the display screen based on the set level. The user interface may also be displayed at a doctor-side terminal.

### CROSS-REFERENCE TO RELATED APPLICATION(S)

Pursuant to 35 U.S.C. §119(a), this application claims the benefit of earlier filing date and right of priority to Korean Patent Application No. 10-2015-0047319, filed in Korea on Apr. 3, 2015, the contents of which are incorporated by reference herein in their entirety.

### BACKGROUND

1. Field

The present disclosure relates generally to a method for generating a user interface and an apparatus for generating a user interface using the method and, more particularly, to a method for generating a user interface and an apparatus for generating a user interface using the method, which changes an interface of a patient-side terminal device and doctor-side terminal device according to patient conditions and provides the changed interface. Also, the method and apparatus facilitates detecting a patient's condition by changing a display layout according to a specific measured value.

2. Background

Methods and apparatuses for generating a user interface of a patient-side terminals and a doctor-side terminals are known. However, they suffer from various disadvantages.

### BRIEF DESCRIPTION OF THE DRAWINGS

The embodiments will be described in detail with reference to the following drawings in which like reference numerals refer to like elements wherein:

FIG. 1 is a diagram illustrating a system for generating a user interface, including a patient-side terminal device, a measurement device, a server, and a doctor-side terminal device according to an embodiment of the present disclosure;

FIG. 2 is a diagram illustrating a configuration of an apparatus for generating a user interface according to an embodiment of the present disclosure;

FIG. 3 is a flow diagram illustrating a method for generating a user interface according to an embodiment of the present disclosure; and

FIG. 4 is a diagram illustrating an example of a user interface according to an embodiment of the present disclosure.

### DETAILED DESCRIPTION

Technical terms used in this specification are used to merely illustrate specific embodiments, and should be understood that they are not intended to limit the present disclosure. As far as not being defined differently, all terms used herein including technical or scientific terms may have the same meaning as those generally understood by an ordinary person skilled in the art to which the present disclosure belongs to, and should not be construed in an excessively comprehensive meaning or an excessively restricted meaning. In addition, if a technical term used in the description of the present disclosure is an erroneous term that fails to clearly express the idea of the present disclosure, it should be replaced by a technical term that can be properly understood by the skilled person in the art. In addition, general terms used in the description of the present disclosure should be construed according to definitions in dictionaries or according to its front or rear context, and should not be construed to have an excessively restrained meaning.

A singular representation may include a plural representation as far as it represents a definitely different meaning from the context. Terms ‘include’ or ‘has’ used herein should be understood that they are intended to indicate an existence of several components or several steps, disclosed in the specification, and it may also be understood that part of the components or steps may not be included or additional components or steps may further be included.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure.

Embodiments of the present disclosure will be described below in detail with reference to the accompanying drawings. Also, the same components or similar components have the same reference number regardless of the figure number, and redundant explanations are omitted.

In describing the present disclosure, if a detailed explanation for a related known function or construction is considered to unnecessarily divert the gist of the present disclosure, such explanation will be omitted, but would be understood by those skilled in the art. The accompanying drawings are used to help easily understood the technical idea of the present disclosure and it should be understood that the idea of the present disclosure is not limited by the accompanying drawings.

Methods and apparatuses for writing and using a PHR (personal health record) are under continual development, and such development has typically focused on methods for writing, collecting, and reading personal health records. For example, one method for constructing and reading a personal health record includes connecting to a computerized patient medical record system such as EMR (electronic medical record) or HER (electronic health record) of a hospital or clinic, or a prescription management system of a drug store. In this method, a personal health record may be constructed, but may enable only reading of limited information from it. Accordingly, even though an individual has read the constructed personal health record, it may be difficult to detect the history or characteristics of a patient's personal health management.

One object of the present disclosure is to provide a method for generating a user interface and an apparatus for generating a user interface using the method, whereby an intuitive display screen can be provided to help a user quickly understand the progress of a disease and the feedback from a medical team.

FIG. 1 is a diagram illustrating a system for generating a user interface according to an embodiment of the present disclosure. The system may include a patient-side terminal device 100, which may be an apparatus for generating a user interface, a measurement device 200, a medical server 300, and a doctor-side terminal device 400. The terminal devices 100 and 400 are referred to herein as patient-side terminal device and doctor-side terminal device simply for ease of description. However, it should be appreciated that these terminals may be any two terminals, for example, a terminal provided at various types of medical service providers or users requiring patient data.

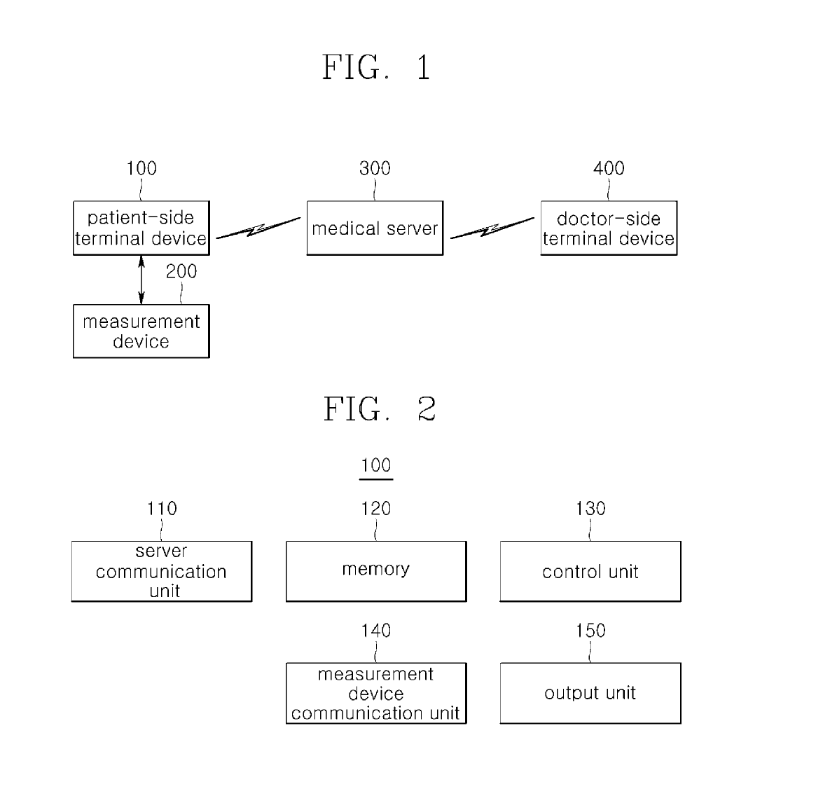

The patient-side terminal device 100 may be an apparatus for generating a user interface that displays a patient health-related history, and may use various types of computing devices configured to receive, process, and output data. A configuration of the patient-side terminal device 100 will be described in detail referring to FIG. 2.

The measurement device 200 may be a device for measuring a patient's health condition. Any devices for measuring a patient's health conditions such as blood pressure, blood sugar, weight, temperature, and the like, may be used. The kind of the measurement device is not limited.

The measurement device 200 may be connected to the patient-side terminal device 100 to transmit the measured values to the patient-side terminal device 100. The measurement device 200 may be a wired or wireless device. The measurement device 200 may be connected with the patient-side terminal device 100 through pairing. Accordingly, when the measurement is completed, the patient health-related data measured by the measurement device 200 may be transmitted to the patient-side terminal device 100. Also, a value for a measurement item that does not need a measurement device may be manually input through the patient-side terminal device 100 by a user.

The medical server 300 may collect and store patient health-related information. The medical server 300 may collect health-related information classified by the patient. The health-related information may be prescriptions, various medical records written in a hospital, a personal health record (PHR), or an electronic health record (EHR), or the like. Also, the medical server 300 may collect and store health-related information classified by the patient from now for a predetermined or specified period of time. For example, the medical server may accumulate the health-related information for a period of years. Moreover, the medical server 300 may be connected to the patient-side terminal device 100 and doctor-side terminal device 400 through a wireline or wireless network so as to transmit and receive the related data.

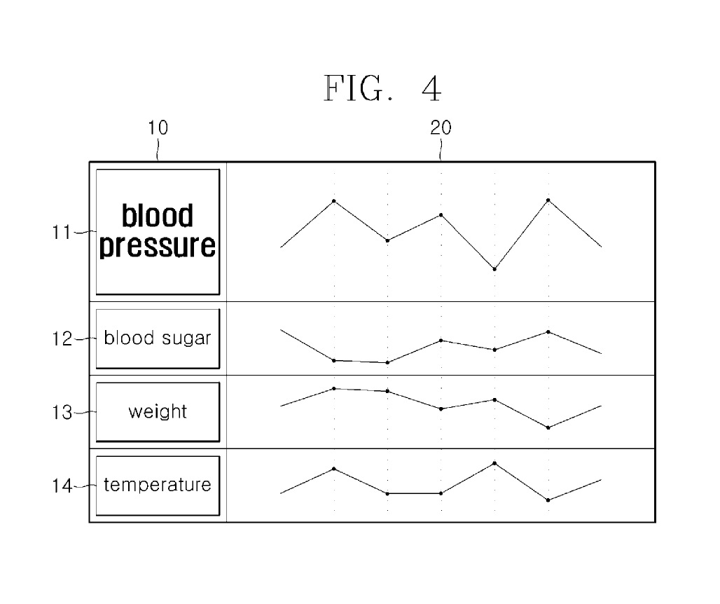

The doctor-side terminal device 400 may transmit and receive various data related to a patient's health from the patient-side terminal device 100 via the medical server 300. Also, according to patient conditions, it is possible to display important measurement data on the doctor-side terminal device 400 to be highlighted to distinguish it from other measurement data. A configuration for displaying important measurement data to be highlighted to distinguish it from other measurement data will be described in further detail with reference to FIG. 4 hereinafter.

FIG. 2 is a diagram illustrating a configuration of an apparatus for generating a user interface according to an embodiment of the present disclosure. The apparatus for generating the user interface may be implemented on the patient-side terminal or the doctor-side terminal. For ease of description, configuration in a patient-side terminal will be described. The patient-side terminal device 100 may include a server communication unit 110, a memory 120, a control unit 130, a measurement device communication unit 140, and an output unit 150.

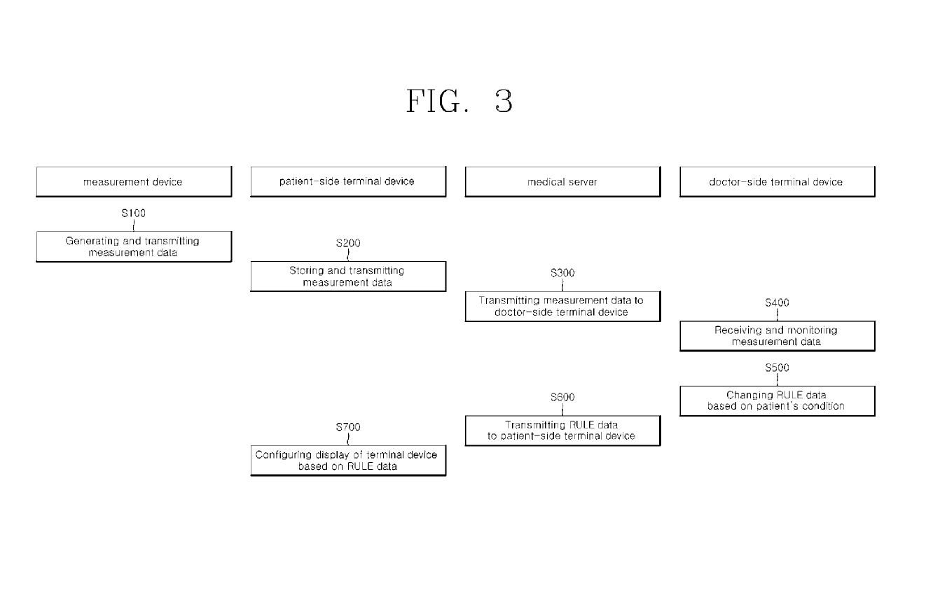

The server communication unit 110 may transmit patient health-related information to a server and receive RULE data. Specifically, the server communication unit may receive various data related to a patient's health from the measurement device 200 paired with the unit, and deliver it to the medical server 300. Also, the medical server 300 may deliver it to the doctor-side terminal device 400. Here, the RULE data may include data for a prescribed set of rules for managing measured patient data and for configuring a display of the measured patient data. The RULE data will be discussed in further detail with reference to FIGS. 3 and 4 hereinafter.

The memory 120 may store and sort at least one record included in the health-related information received from the measurement device 200 paired with the server communication unit 110. The memory 120 may store the measurement data in chronological order. Also, the memory 120 may store a program for controlling the patient-side terminal device 100.

The control unit 130 may generate a user interface screen for displaying information. According to a predetermined standard, the control unit 130 may display a list of measurement devices determined to be necessary based on the name of the patient's disease, a state of pairing with the measurement device 200, result values measured by the measurement device 200, and the like.

The control unit 130 may display at least one measurement device 200 according to a patient condition. If the patient suffers from multiple diseases, the control unit may display at least one measurement device 200 needed to check the progress of each disease. Also, when the measurement device 200 is selected, it is possible to check previous data measured by the selected device.

Also, when displaying measurement data of a specific measurement device 200, multiple data may be displayed at the same time. Accordingly, it is possible to easily detect the progress and state of the disease and patient condition.

Also, the control unit 130 may highlight data measured by the specific measurement device 200. The highlighted measurement data may be changed according to a configuration set by a user. For example, an important measurement device or type of data may be differentiated from other measurement devices or data by displaying the measurement data in a different color, a different size, or in special characters, etc., but the format is not limited.

The measurement communication unit 140 may be a component to enable pairing with a measurement device 200. Through the pairing, the patient-side terminal device 100 may receive data from the measurement device 200. Also, through the pairing, the measurement device 200 may read a configuration value of the measurement device from the patient-side terminal device. Accordingly, the measurement device 200 may be operated to adapt to the configuration values according to a particular patient.

The output unit 150 may display the user interface screen generated by the control unit 130. The output unit may include a display unit, and the display unit may display the user interface screen generated by the control unit 130. The display unit may be combined with a user input means such as a touch screen. Also, an order or request from the user, which is input through the touch screen, may be delivered to the control unit 130. Although the output unit 150 is described herein as a display unit, the present disclosure is not limited thereto, and may include various types of output devices according to the intended application and desired functions.

FIG. 3 is a flow diagram illustrating a method for generating a user interface according to an embodiment of the present disclosure. In step S100 shown in FIG. 3, a patient-side terminal device may pair with the measurement device 200 and receives the patient health-related information from the measurement device 200.

By pairing, the patient-side terminal device 100 may receive measurement data from the measurement device 200, and options or settings for the measurement device may be set depending on the configuration values of the measurement device, which may be set differently according to the patient.

The health-related information may be data measured by the measurement device, and may be stored by the patient-side terminal device 100. Accordingly, the history of treatments and prescriptions, variation of data, etc., may be checked.

Also, the measurement data may be displayed on the patient-side terminal device 100. The measurement data may be displayed on the patient-side terminal device 100 based on RULE data previously set, for example, by a medical team. Even though measurement data may be the same, a layout of the screen displayed on the patient-side terminal device 100 or doctor-side terminal device 400 may be different if the RULE data is changed.

Next, the patient-side terminal device 100 may store the received measurement data and transmit it to the medical server 300, in step S200, and the medical server 300 may transmit the received measurement data to the doctor-side terminal device 400, in step S300. The doctor-side terminal device 400 may receive and monitor the data, in step S400, and may change the RULE data according to the patient conditions, in step S500. The RULE data may be changed according to a previously set table, by the medical team, or the like. A more detailed explanation will be described later referring FIG. 4. Next, the RULE data may be transmitted from the doctor-side terminal device 400 to the patient-side terminal device 100 via the medical server 300, in step S600, and the patient-side terminal device 100 may configure a display screen according to the received RULE data, in step S700.

FIG. 4 is a diagram illustrating an example of a user interface according to an embodiment of the present disclosure. Measurement items 10 and measurement values 20 may be displayed on the user interface screen. The measurement values may be sorted chronologically. As measurement values for each measurement item may be sorted according to the same time scale, relationships with other measurement items measured at the same time may be displayed to enable the user to more easily review the data.

In one embodiment, the measurement items 10 may include blood pressure 11, blood sugar 12, weight 13, and temperature 14, but the type of measurement items may be differently configured according to a type of disease or the progress of the disease or patient.

Each measurement item 10 may be displayed using the same icon, size, color, and form, or displayed to distinguish one measurement item from other measurement items. Each measurement item 10 may be assigned a particular level among multiple levels according to the measurement values. For example, the multiple levels may be classified into five levels, and each measurement item may be configured to have the same number of level.

For example, the level of measurement value for each item may correspond to a level indicating a stable condition or a level indicating a serious condition. When the measurement value is outside a stable range, the level may be set to be higher to indicate a serious condition.

For example, if the measured maximum blood pressure 11 is more than 140 and less than 145, the level for blood pressure 11 may be set to level 4. If the measured maximum blood pressure 11 is more than 145, it may be set to level 5. Also, low blood pressure may be monitored and assigned a level. For example, if the measured minimum blood pressure 11 is more than 60 and less than 70, it may also be set to level 4.

Each measurement item 10 may be set to have a higher level value according to how far outside the normal range a particular measurement resides. For example, when blood pressure on the high side is set to level 3 according to a value of measured maximum blood pressure, and the blood pressure on the low side is set to level 4 according to a value of measured minimum blood pressure, the level for the blood pressure 11 measurement item may be finally set to have the higher of the two level values (i.e., level 4).

Also, when the measurement value goes beyond a predetermined range, it is possible to trigger an alarm in the doctor-side terminal device 400 and to highlight the measurement data on their terminal as well as on the patient-side terminal device 100. Also, considering a height of the patient and progress of the disease, a normal range for weight may be previously set, and the higher the absolute value of the difference between the normal range and measurement value, the higher the level value may be set. These customizations may be accomplished using the RULE data.

The RULE data may be data corresponding to a set of rules for managing the measured data as well as setting a display format of the measured data. The RULE data for displaying a level value that each measurement value has may be previously set. Also, the RULE data may be differently set or customized depending on the patient or time periods. Accordingly, the same measurement values may be set to different levels according to certain RULE data.

Level values for the respective measurement items 10 may be added up, and the display screen may be differently configured according to a ratio of the level value to the sum of the level values. One of the measurement items 10 having a relatively high level value may be displayed in a larger size or deeper color than the measurement items 10 having a relatively low level value.

For example, if the measured level value for blood pressure 11, blood sugar 12, weight 13, and temperature 14 is 4, 3, 3, and 2, respectively, the sum of the measured level values is 12. According to the RULE data related to the display method reflecting the respective measured level, as the measured level value for blood pressure 11 is 4, and the sum of the measured level values is 12, blood pressure may be displayed in size corresponding to 4/12, or ⅓, of the whole display screen of the device. Similarly, blood sugar 12 and weight 13 may be displayed in size corresponding to ¼ of the whole display screen, and temperature 14 may be displayed in size corresponding to ⅙ of the whole display screen. Accordingly, each measurement item 10 may be displayed according to its particular level value. On the other hand, if the RULE data prescribes that the measurement item having the highest measured level is to be displayed in size corresponding to a prescribed ratio (e.g., ½) of the whole display screen of the device while the remaining measurement items are displayed in the same size to fill the remaining portion (e.g., ½) of the whole screen, blood pressure 11 may be displayed in size corresponding to ½ of the whole display screen of the device, and the other measurement items 12, 13, 14 may each be displayed in size corresponding to ⅙ of the whole display screen. Accordingly, as an item with a lower level is displayed in a smaller size while an item with a higher level is displayed in a larger size, intuitive understanding of the data is possible.

As described above, the layout of the screen, which is differently displayed depending on the level, has an effect of intuitive understanding. One object of the present disclosure is to provide a method for generating a user interface and an apparatus for generating a user interface using the method, whereby an intuitive display screen can be provided to help a user quickly understand the progress of a disease and the feedback from a medical team.

A method for generating a user interface according to an embodiment of the present disclosure may include: receiving respective data for at least one measurement item associated with a patient's health from a measurement device; setting a level for the respective data based on RULE data; and changing a layout of a display screen based on the level.

An apparatus for generating a user interface according to an embodiment of the present disclosure may include: a server communication unit for receiving patient health-related data measured by a measurement device designated on a screen, based on the name of the patient's disease; a measurement device communication unit for paring with the measurement device; a memory for storing and sorting the data; and a control unit for setting a level for the respective data and changing a layout of a display screen according to the set level.

A method for generating a user interface and an apparatus for generating a user interface using the method according to an embodiment of the present disclosure provides an intuitive display screen, and thus helps a user quickly understand the progress of a disease and the feedback from a medical team.

The embodiments according to the present disclosure as described above may be implemented in the form of program instructions that can be executed by various computer components, and may be stored on a computer-readable recording medium. The computer-readable recording medium may include program instructions, data files, data structures and the like separately or in combination. The program instructions stored on the computer-readable recording medium may be specially designed and configured for the present disclosure, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler or the like, but also high-level language codes that can be executed by a computer using an interpreter or the like. The above hardware devices may be changed to one or more software modules to perform the operations of the present disclosure, and vice versa.

The particular implementations shown and described herein are illustrative examples and are not intended to otherwise limit the scope of this disclosure in any way. For the sake of brevity, conventional electronics, control systems, software development, and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the embodiments unless the element is specifically described as “essential” or “critical”.

The use of the terms “a,” “an,” “the,” and similar referents in the context of describing the embodiments (especially in the context of the following claims) are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. The steps of all methods described herein can be performed in any suitable order unless otherwise indicated herein or otherwise clearly contradicted by context. The examples provided herein and the exemplary language (e.g., “such as” or “for example”) used herein are intended merely to better illuminate the embodiments and does not pose a limitation on the scope of this disclosure unless otherwise claimed. In view of this disclosure, numerous modifications and adaptations will be readily apparent to those skilled in this art without departing from the spirit and scope of this disclosure.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A method for generating a user interface for displaying patient health-related data, comprising:
establishing a connection between a patient-side terminal and at least one measurement device;
receiving, at the patient-side terminal, measured patient data from the at least one measurement device for at least one type of measurement related to patient health;
setting, at the patient-side terminal, a level for the measured patient data based on data for a prescribed set of rules;
displaying the measured patient data on a display screen; and
changing a layout of the displayed patient data on the display screen based on the set level.

2. The method of claim 1, wherein the level is one among a range of levels, each of the at least one type of measurement corresponding to the same range of levels.

3. The method of claim 1, wherein the level is set to a higher level according to an amount in which a value for the measured patient data is outside a prescribed range.

4. The method of claim 1, further comprising transmitting the measured patient data to a medical service provider-side terminal.

5. The method of claim 1, further comprising triggering an alarm in a medical service provider-side terminal when a value of the measured patient data is outside a prescribed range.

6. The method of claim 1, further comprising highlighting the measured patient data when a value of the measured patient data is outside a prescribed range.

7. The method of claim 1, wherein, when the level of the measured patient data for a prescribed type of measurement is higher than a prescribed level, the measured patient data is displayed in a larger size than when the level of measured patient data for the prescribed measurement item is lower than or equal to the prescribed level.

8. The method of claim 7, wherein a plurality of types of measurements related to patient health is displayed on the display screen, a display size of each of the plurality of types of measurements being based on a ratio between a set level for a prescribed type of measurement to set levels of all of the plurality of types of measurements.

9. The method of claim 1, wherein the type of measurement includes at least one of blood pressure, blood sugar, heart rate, weight, or temperature.

10. The method of claim 1, wherein the measured patient data includes a first type of measurement and a second type of measurement, data for the first type of measurement and data for the second type of measurement having a plurality of data points measured over a prescribed time period.

11. The method of claim 10, wherein the data points for the first type of measurement and the data points for the second type of measurement are displayed on the display screen to share a common time scale.

12. The method of claim 1, wherein the data for the prescribed set of rules includes a different set of rules according to a patient and a time period associated with the measured data.

13. The method of claim 1, wherein the data for the prescribed set of rules to set the level is based on at least one of a type of illness, a condition of the patient, age, sex, or a time associated with data measurement.

14. A computer-readable recording medium having recorded thereon a program for executing the method of claim 1.

15. An apparatus for generating a user interface for displaying patient health-related data, comprising:
a display screen;
a server communication unit configured to communicate with a server;
a measurement device communication unit to pair with at least one measurement device;
a memory to store data received from the server and the at least one measurement device; and
a control unit configured to generate a user interface to display patient health-related data,
wherein measured patient data is received from the at least one measurement device according to at least one type of measurement related to patient health, a level for the measured patient data is set based on data for a prescribed set of rules associated with the measured patient data, the measured patient data is displayed on the display screen, and a layout of the displayed patient data on the display screen is changed based on the set level.

16. The apparatus of claim 15, wherein the server communication unit transfers data to generate an alarm at a medical service provider-side terminal when a value of the measured patient data is outside a prescribed range.

17. The apparatus of claim 15, wherein, when the level of the measured patient data for a prescribed type of measurement is higher than a prescribed level, the measured patient data is displayed in a larger size than when the level of measured patient data for the prescribed measurement item is lower than or equal to the prescribed level.

18. The apparatus of claim 17, wherein a plurality of types of measurements related to patient health is displayed on the display screen, a display size of each of the plurality of types of measurements being based on a ratio between a set level for a prescribed type of measurement to set levels of all of the plurality of types of measurements.

19. The apparatus of claim 15, wherein the measured patient data includes a first type of measurement and a second type of measurement, data for the first type of measurement and data for the second type of measurement having a plurality of data points measured over a prescribed time period.

20. The apparatus of claim 19, wherein the data points for the first type of measurement and the data points for the second type of measurement are displayed on the display screen to share a common time scale.
